(19) United States
(12) Patent Application Publication
(10) Pub. No.: US 2009/0004128 A1
(43) Pub. Date: Jan. 1, 2009

(54) Non-Irritant Rapid-Action Dipilatory Composition

(75) Inventor: Michel Flork, Saint Martin D'Ardeche (FR)

Correspondence Address: BROWDY AND NEIMARK, P.L.L.C.;624 NINTH STREET, NW, SUITE 300, WASHINGTON, DC, 20001-5303, US

(73) Assignee: PRODUCCIONES INFOVISION, Mexico (MX)

(21) Appl. No.: 11/718,306

(22) PCT Filed: Oct. 24, 2005
(86) PCT No.: PCT/FR05/02642

§ 371 Date: Jul. 11, 2008

(30) Foreign Application Priority Data
Oct. 29, 2004 (FR) 0411594

(51) Int. Cl. A61K 8/46; A61K 8/23; A61Q 9/04

(52) U.S. Cl. 424/70.1

(57) ABSTRACT

The invention relates to a rapid-action depilatory composition, comprising at least one bisulphic and/or sulphite compound and an organic reducing agent in a cosmetically acceptable medium.

The invention relates to a non-irritant rapid-action depilatory composition comprising as active ingredient at least one bisulphite and/or sulphite compound and an organic reducing agent, in a cosmetically-acceptable medium.

The depilation mechanism requires breaking the bonds which ensure cohesion between the keratin molecules which constitute the hair. These bonds are constituted, on the one hand, by disulphide bridges existing between two cysteine units of the keratin molecules, forming cystine units, and on the other hand, by hydrogen bonds which maintain the peptide bonds between themselves.

Organic reducing agents containing sulphur, such as for example the mercaptocarboxylic acids of formula HS—(CH₂)ₙ—COOH in which n is an integer from 1 to 4, in particular thioglycolic acid (HS—CH₂—COOH), are known for their degradation action vis-à-vis keratin and are in particular used in compositions for perming hair.

The reaction between keratin and the sulphurated organic compound is shown in the diagram below, in which R—SH represents the sulphurated organic compound and X represents the part of the keratin molecule which does not contain sulphur: R—SH+X—S—S—X→R—S—S—X+RS⁻+H⁺

As indicated above, this type of compounds leads to breakage of the disulphide bridges of the keratin, converting the cystine units to cysteine units, then the formation of new disulphide bridges between the free SH groups of the cysteine and those of the thioglycolic acid, making it possible to set the hair in the desired shape.

When one wishes to obtain a depilatory action, it is sought, on the one hand, to avoid the formation of the these new bonds and, on the other hand, to break the hydrogen bonds. This breakage is carried out by a massive supply of OH⁻ ions, which leads to an increase in the pH.

The increase in the pH encourages the formation of RS⁻ compounds and the following reaction: RS⁻+X—S—S—X→XS⁻+R—SS—X takes place.

However, it is noted that at alkaline pH (greater than 7), and in particular at pHs comprised between 7 and 12.7, namely the pH authorized by the European standard in force for hygiene products which can be used on the skin, an undesirable increase of R—SS—X complexes was obtained, which cannot be compensated for by an increase in the concentration of R—SH compound given the standards in force.

Moreover there exists an increasing demand by consumers for a reduction in the exposure time required for the depilatory composition, with the aim of obtaining a product which is both more active and causes less skin irritation.

Depilatory compositions containing alkaline or alkaline-earth salts of thioglycolic acid, guanidine, and, sodium sulphite as antioxidant, are described in the U.S. Pat. No. 3,154,470.

However, the use of sodium sulphite, having a high solubility in a medium containing a strong base such as Ca(OH)₂, leads to an increase in the pH of the composition, which is highly damaging to its stability.

The technical problem to be resolved therefore consists of providing a stable depilatory composition which has a rapid action while being non-irritant to the skin, and this composition must also fall within the framework of the standards in force.

It has now been found that it is possible to obtain a rapid and non-irritant action, both thanks to the high pH of the composition and to the reduction of the exposure time, by introducing into the depilatory composition at least one bisulphite and/or sulphite compound and an organic reducing agent, in a cosmetically-acceptable medium.

In fact, it has been found that, advantageously, the reducing action of the bisulphite and/or sulphite compound vis-à-vis the cystine units of the keratin anticipated the action of the organic reducing agent, and encouraged the reaction of the free SH groups of the cysteine units with said reducing agent, thus making possible the rapid solubilization of the keratin, leading to hair loss.

According to a first aspect, the invention therefore relates to a rapid-action depilatory composition comprising at least one bisulphite and/or sulphite compound and an organic reducing agent, in a cosmetically-acceptable medium.

Said composition is intended to be applied on human or animal skin.

Advantageously, it has also been found that the composition according to the invention could have an improved depilatory activity when its acid components are found in the form of a salt with a strong organic base, having a pK_{B} of the order of approximately 6 to 13.5, such as for example guanidine, hydrazine or hydroxylamine.

In fact, although the combination of an organic reducing agent and at least one sulphite or bisulphite compound already confers an improved depilatory activity on the composition according to the invention, these strong organic bases are capable of increasing the denaturing power of said composition vis-à-vis proteins and thus facilitating the activity of the reducing agent.

Said strong organic base salts, in particular the guanidine salts, are novel products which represent another subject of the invention.

According to a first aspect of the invention, the bisulphite and/or sulphite compound is a compound of formula M₂S₂O₅ or M₂SO₃, in which M represents an alkaline cation, such as, for example, potassium, calcium, lithium, barium or strontium.

Alternatively, the bisulphite and/or sulphite compound is in the form of an alkaline salt with a strong organic base, having a pK_{B} of the order of approximately 6 to 13.5, such as for example guanidine, hydrazine or hydroxylamine.

The bisulphite or sulphite salts with said bases, in particular guanidine are novel products which represent a subsequent subject of the invention.

Alternatively, said bisulphite and/or sulphite compound can be replaced by sulphur dioxide SO₂.

Advantageously, a bisulphitic compound of aldehyde or ketone will be used, such as the bisulphitic compounds of acetaldehyde, propionaldehyde, butyraldehyde, benzaldehyde, glyoxal, glyoxylic acid. These compounds are preferentially in the form of sodium, potassium, strontium, calcium, magnesium, arginine salts, or in the form of a strong base salt having a pK_{B} of the order of approximately 6 to 13.5, such as for example guanidine, hydrazine or hydroxylamine.

Preferably, the depilatory composition according to the invention comprises from 0.1 to 25%, preferably from 1 to 10%, in particular approximately 5% of bisulphite and/or sulphite compound, by weight of the total composition.

According to the invention, the depilatory composition also comprises an organic reducing agent. This can, for example, be chosen from sulphurated compounds such as dithiothreitol, thioglycerol, thioglycol, thiosalicylic acid and its salts, N-acetyl-L-cysteine, NaHSO₃, LiS, Na₂S, K₂S, MgS, CaS, SrS, BaS, (NH₄)₂S, sodium 6-8-dithiooctanoate dihydrolipoate, sodium 6-8-dithiooctanoate, hydrogen sulphide salts, the mercaptocarboxylic acids of formula HS—(CH₂)ₙ—COOH in which n is an integer from 1 to 4 and their salts, thiomalic acid and its salts, thiomaleic acid and its salts, thiolactic acid and its salts, dihydrolipoic acid and its salts, homocysteine, cysteine, cysteamine, glutathione, thioglycolamide, 1-,3-dithiopropanol, keratinase, hydrazine, hydrazine sulphate, hydrazine disulphate, triisocyanate or guanidine. The preferred reducing agents are chosen from hydrazine, hydroxylamine, thiourea, thioacetamides and the mercaptocarboxylic acids of formula HS—(CH₂)ₙ—COOH in which n is an integer from 1 to 4, in particular thioglycolic acid, and their salts.

Advantageously, said reducing agents, in particular the mercaptocarboxylic acids, are present at the rate of 0.2% to 5%, preferably approximately 5%, by weight of the total composition, thioglycolic acid being preferred.

For the preparation of the composition according to the invention, when the reducing agent is in acid form, an alkaline salt of said acid will preferably be used. According to an advantageous aspect, a strong organic base salt, having a pK_{B} of the order of approximately 6 to 13.5 is used, such as for example guanidine, hydrazine or hydroxylamine.

The strong organic base salts of said acids, in particular the guanidine salts of mercaptocarboxylic acids, and in particular the guanidine salts of thioglycolic acid, are novel products which represent a subsequent subject of the invention.

The pH of the composition according to the invention is preferably comprised between 9 and 12.7, in particular of the order of 12.3. The pH can be adjusted in said composition using an alkaline agent which is usual in the field, for example calcium hydroxide.

The depilatory composition according to the invention can also comprise the usual components of such a composition, namely in particular at least a compound chosen from:
surface active agents, such as for example a lauryl sulphate compound, in particular sodium lauryl sulphate or a salt of lauryl sulphuric acid with a strong organic base as defined above, in particular the lauryl sulphate of guanidine, hydrazine or hydroxylamine, preferably at the rate of 0.1 to 5% by weight of the total composition,
anti-irritant agents, such as, for example, glycerrhetinic acid (also called enoxolone), salicylic acid or one of its salts, in particular a strong organic base salt as defined above, in particular the salicylate of guanidine, hydrazine or hydroxylamine, or vitamin E, preferably at the rate of 0.01 to 5% by weight of the total composition,
agents which accelerate the reduction reaction of keratin, such as for example urea or thiourea, preferably at the rate of 0.1% to 15% by weight of the total composition,
pH regulators, such as for example calcium hydroxide or guanidine base, preferably at the rate of 0.1 to 10% by weight of the total composition,
sequestering agents of monovalent or divalent cations, such as for example sodium gluconate or a salt of gluconic acid with a strong organic base as defined above, in particular the gluconate of guanidine, hydrazine or hydroxylamine, preferably at the rate of 0.5 to 20% by weight of the total composition.

Other additives generally used in the field of cosmetics or hygiene can also be present in the composition according to the invention, in particular preservatives, antioxidants or perfumes which are well known in these technical fields.

A person skilled in the art is in a position to choose, from all of these possible additives, both the composition and the quantity of those which are to be added to the composition, so that it retains all of its properties.

The invention also relates to, according to a subsequent aspect, a method for the preparation of the depilatory composition described above.

Said method comprises the steps consisting of:
preparing a water-in-oil emulsion which does not contain the organic reducing agent and the sulphite or bisulphite compound,
preparing an aqueous suspension containing the organic reducing agent and the sulphite or bisulphite compound,
introducing said suspension into the oil-in-water emulsion, and
subjecting the mixture to shearing.

The depilatory composition is preferably presented in the form of a gel, foam, emulsion or cream.

The invention also relates to a method for removing superfluous hair, comprising the steps consisting of:
applying a sufficient quantity of depilatory composition as described above onto a skin surface comprising said hairs, then
removing said composition by wiping or rinsing, this action having the effect of simultaneously removing the hairs.

Advantageously, the composition according to the invention can be used effectively as a thin film, with a thickness of the order of 1 mm, even 0.1 to 0.2 mm. In addition, the composition once spread in the form of a thin film can be moved and repositioned without loss of activity.

Preferably, the wiping or rinsing of the composition is carried out after a period ranging, for example, from approximately 1 to 5 minutes after the application of the depilatory composition.

The wiping can, for example, be carried out using an absorbent paper or tissue or also a scraping instrument designed for this purpose. The rinsing can be, for example, carried out with water.

The invention also relates according to a subsequent aspect, to novel salts of strong organic bases, having a pK_{B} of the order of approximately 6 to 13.5, such as for example guanidine, hydrazine or hydroxylamine, as well as their preparation method.

Said method comprises the steps consisting of
reacting a strong acid with a strong organic base carbonate as defined above, and
degassing the carbon dioxide formed.

The strong organic base used is preferably chosen from guanidine, hydrazine or hydroxylamine, guanidine being preferred.

By strong acid, is meant an acid having a pK_{A} less than 6.

The reaction is preferably carried out at a temperature of 0 to 80° C.

As indicated above, such strong organic base salts can be used as components of the composition according to the invention.

Preferred compounds for the purposes of the invention are guanidine mercaptocarboxylic acid salts, in particular guanidine thioglycolate and diguanidine thioglycolate; gluconic acid salts, in particular guanidine gluconate; salicylic acid salts, in particular guanidine salicylate; lauryl sulphuric acid salts, in particular guanidine lauryl sulphate; hydrochloric acid salts, in particular guanidine hydrochloride; and formic acid salts, in particular guanidine formate.

The examples below illustrate the invention non-limitatively. Except where otherwise indicated, the percentages are shown as percentages by weight.

### EXAMPLE 1

### Depilatory Composition

The following composition was prepared:

| Ingredients | Quantity (% by weight) |
|---|---|
| Cetyl alcohol/Ceteth-20/Steareth-20 | 2.00 |
| (Emulcire ® 61WL2659, Gattefossé) | |
| Glyceryl stearate/PEG-75 stearate | 2.90 |
| (Gelot ® 64, Gattefossé) | |
| hydrogenated palm oil | 0.41 |
| castor oil | 0.22 |
| octyldodecyl myristate | 1.85 |
| seppicide hb (Seppic) | 0.26 |
| butyl hydroxytoluene | 0.06 |
| propylene glycol | 1.79 |
| urea | 9.88 |
| purified diethylene glycol monoethyl | 3.26 |
| ether | |
| calcium sulphite | 5.22 |
| thioglycolic acid | 4.94 |
| guanidine base | 7.57 |
| water | 54.41 |
| calcium hydroxide | 0.5 |
| gluconic acid | 2.96 |
| Lauryl sulphuric acid | 0.22 |
| enoxolone | 0.12 |
| salicylic acid | 1.22 |
| vitamin E | 0.21 |
| Total | 100 |

This composition was produced as follows:

An oil phase is prepared containing the mixture Cetyl alcohol/Ceteth-20/Steareth-20, glyceryl stearate/PEG-75 stearate, hydrogenated palm oil, castor oil, octyldodecyl myristate, seppicide HB and butyl hydroxytoluene. Then an aqueous phase is prepared containing water, propylene glycol and urea. The oil phase is heated to a temperature of 65° C., then the aqueous phase is introduced and the mixture emulsified in an emulsifier (Laborpilot IKA) with a speed of rotation of 13700 rpm and a shear rate of 40 m/s. A thick emulsion is thus obtained to which is added at approximately 35° C. the purified diethylene glycol monoethyl ether in which the perfume, then the vitamin E, has been dissolved.

Then an aqueous solution is prepared containing water, guanidine thioglycolate, guanidine gluconate, guanidine lauryl sulphate and guanidine salicylate. The calcium sulphite in suspension and the calcium hydroxide are added to this solution in order to adjust the pH to a value of approximately 12.3 to 12.4.

The suspension thus obtained is introduced into the emulsion obtained previously, and the mixture is subjected to shearing at 40 m/s while maintaining the temperature at 32° C.

A depilatory composition according to the invention is thus obtained, having a stability at ambient temperature which is equal to or greater than 6 months.

### EXAMPLE 2

### Study of the Depilatory Activity

1/ Description of the Test

a) Measurement of the Diameter of the Hairs

Hairs are collected from the leg close to the tibia.

The diameter of the hairs is measured with the Palmer Digital (Mitutoyo Digimatic micrometer) with a precision to the μm. The reproducible values vary from 40 to 50 μm.

Verification by microscope (Olympus U-CMAD3 with Pixelink camera and Morpho Lite software from Explora Nova) confirms the diameters measured while showing that the hairs are not cylindrical but ovoid, and that the Palmer device is reading the maximum diameter. This is probably due to a twist in the hair during measurement with the Palmer device, which locates the largest diameter within the measured length (6 mm).

The average value of all the readings is 46 μm.

By way of comparison, measurements carried out under the same conditions on “normal” hairs give an average value of 73 μm, while for those carried out on “fine” hairs, the value is 40 μm.

In order to extend the times necessary to reach breaking point, the test is carried out with “normal” hairs.

b) Test

A hair is inserted in a 200 μm hole. This hole is provided in a plastic plate 2 mm thick, with a depression 0.5 mm deep centred on the upper part of the hole. This depression is filled with a drop of the solution or cream to be tested.

A 10 g weight (in the form of a lead clip) is fixed to the base of the hair, and a similar clip holds it on top of the plate. The hair, with a clip on each end is held vertical with a tension of 10 g.

When a drop of the reagent to be tested is added to the depression provided for this purpose, a chronometer is started in order to determine the time period required for complete breakage, with the weight falling and the hair remaining.

2/ Results

A commercial cream containing thioglycolic acid (Veet® Raséra-Reckitt & Benkiser) is tested under the conditions described below.

The ingredients of the Veet® Raséra cream, as indicated on the commercial packaging in decreasing order of their concentration, are the following:

Water

Urea

Potassium thioglycolate

Calcium hydroxide

Cetearylic alcohol

Ceteareth-20

PPG-15 Stearyl ether

magnesium trisilicate

potassium hydroxide

propylene glycol

sodium, magnesium, lithium silicate

liquid paraffin

prunus amydalus dulcis oil

acrylate copolymer

silk amino acids

sodium gluconate

hydrated silica

perfume

citronellol hexyl cinnamal

butylphenyl propional

An average time is obtained based on 10 readings of 10 min 13 s. The pH of this cream is 12.7 at 20° C. by direct reading and of 13.3 at 20° C. by 1% dilution in distilled water (this is due to undissolved calcium hydroxide in the initial cream).

7% potassium sulphite is added to this same cream and mixing is carried out with a rotor agitator at 24,000 rpm; An average value is obtained based on 10 tests of 7 min and 19 s, with a pH of 12.2.

After the addition of 0.1 ml soda at 30% concentration to the previous sample, the pH is readjusted to 12.7 at 20° C. in order to take measurements under the same pH conditions as those of the commercial cream. An average measured value of 1 min 19 s was thus obtained.

All these tests were carried out on a single hair.

Tests carried out by adding guanidine sulphite instead of potassium sulphite to the Veet® Rasera cream have made it possible to obtain lower average values than those obtained by the addition of potassium sulphite.

### EXAMPLE 4

### Study of the Depilatory Activity

The test described above was carried out with different depilatory compositions on a hair with a diameter of 60 μm.

The results are shown in Table 1 below.

TABLE 1

| | | Potassium | | Time period |
|---|---|---|---|---|
| | Thioglycolic | bisulphite | | for complete |
| Test No. | acid % | % | pH | breakage |
| 1 | 5 | | 12.7 | 8 to 10 min |
| 2 | 5 | 5 | 12.7 | <2 min |
| 2 | 4 | | 12.7 | 10 to 12 min |
| 4 | 4 | 4 | 12.7 | <2 min 30 s |

The results show that the addition of potassium bisulphite significantly reduces the time period necessary for complete breakage of the hair.

### EXAMPLE 5

### Study of the Depilatory Activity

A commercial cream containing thioglycolic acid (Veet® Rasëra) is applied to a leg in a layer approximately 1 mm thick and this same cream with 7% bisulphite added to it is applied to the other leg. It is noted that the depilatory action is obtained after 90 seconds for the cream with the added bisulphite, while 2 minutes is not enough time for the commercial cream to achieve a depilatory action.

### EXAMPLE 6

### Study of the Depilatory Activity

The composition of Example 1 in the form of cream was compared with a commercial cream containing thioglycolic acid (Veet® Raséra).

Method:

A brown hair removed extemporaneously from the leg at the level of the tibia is placed on a glass plate immediately after its extraction. The chronometer is started during the deposit of a micro-drop of cream on the hair, at more than 2 mm from the root. A glass slide cover is then put in position and the assembly is placed under the microscope (OLYMPUS BX41).

The time is taken at the moment of break, then at the time of total dissolution.

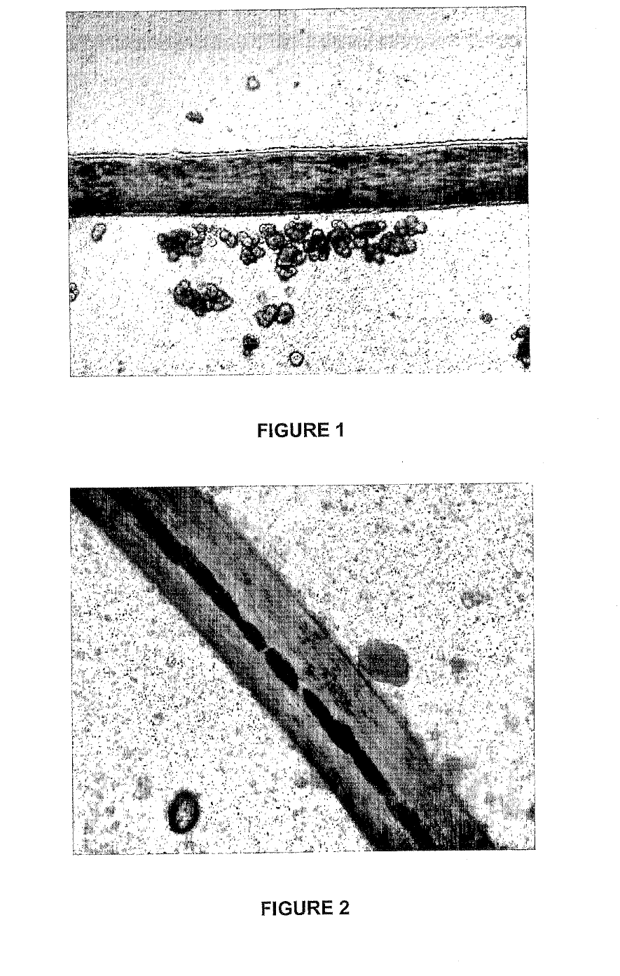

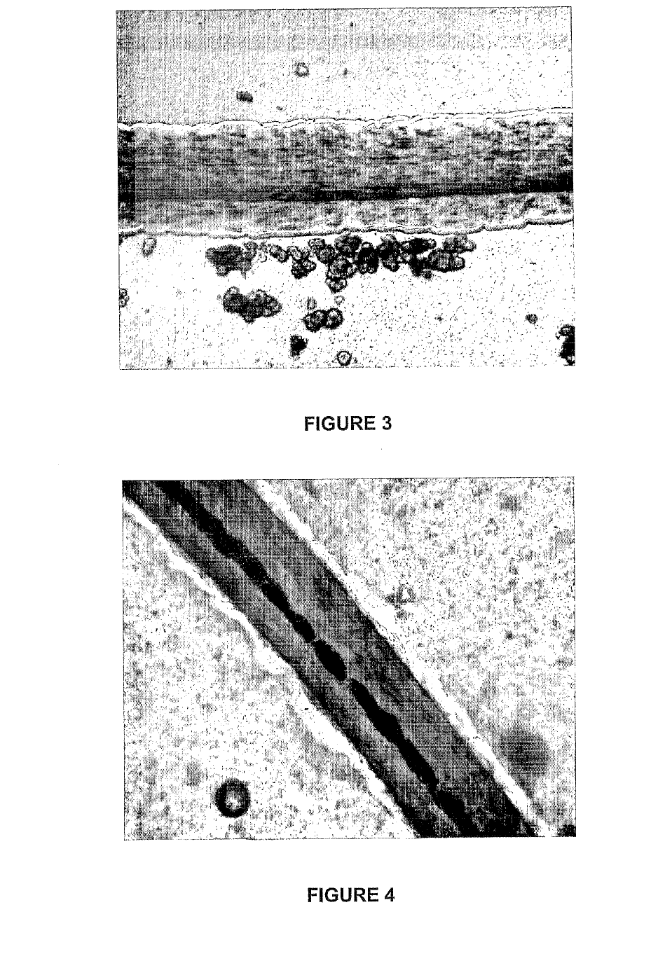

The photos obtained show the breaking of the hair, then the complete dissolution of same, without any mechanical action. These are shown in FIGS. 1 to 7, in which:

FIGS. 1, 3 and 5 represent the hair immersed in the composition according to the invention at the respective times of 18 s, 120 s and 252 s (magnification 10×40);

FIGS. 2, 4, 6 and 7 represent the hair immersed in the Veet® cream for the respective times of 18 s, 120 s, 252 s and 780 s (magnification 10×80);

In the photos at 18 s (FIGS. 1 and 2), a slight attack will be noted on the cuticle of the hair with the formation of a fine white layer.

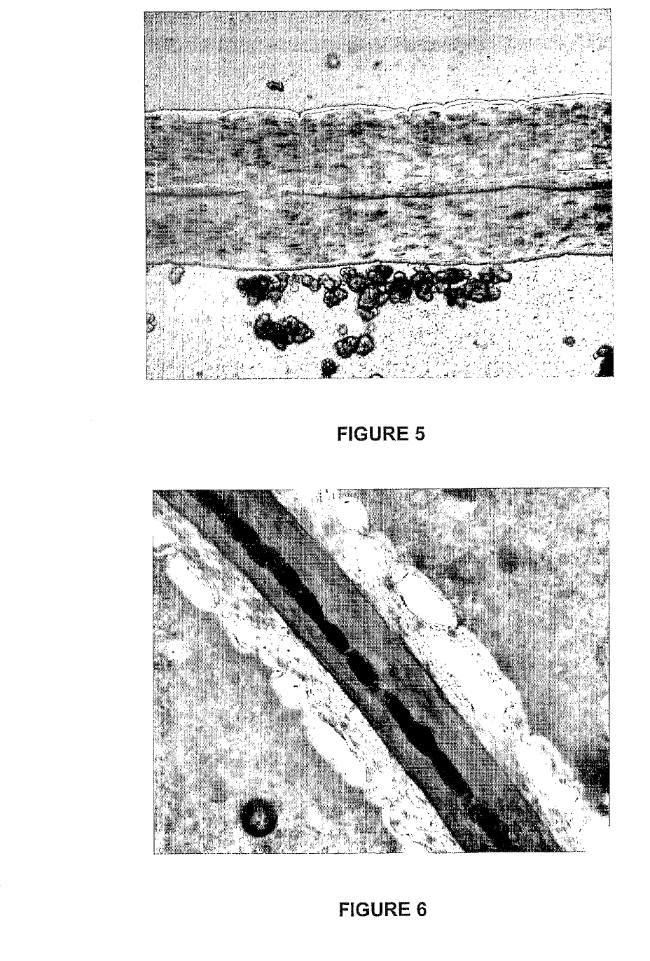

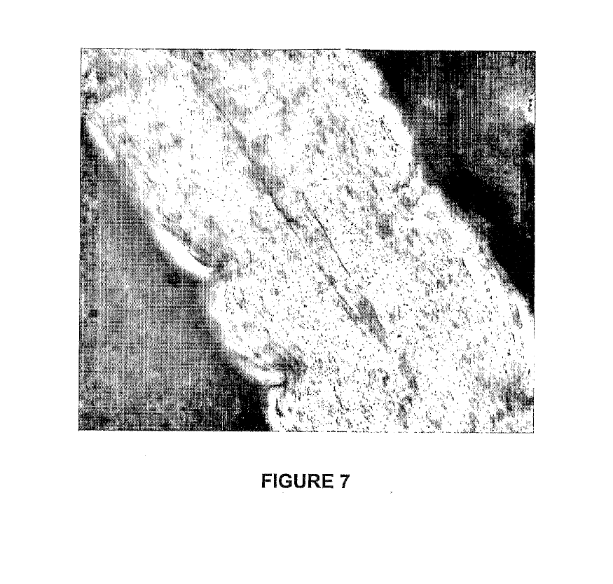

Then in the following photos, the solubilized proteins spread out and the width seems to increase, while the hair, still strong, is thinned, to reach breaking point at 252 s for the cream according to the invention (FIG. 5) and 780 s for the cream Veet® (FIG. 7).

The results show that the presence of sulphite compound in the depilatory cream allows the action of the thioglycolic acid to be maintained, while its absence causes a slowing of the activity due to depletion in active thioglycolic acid.

### EXAMPLE 7

### Preparation of Guanidine Thioglycolate

1801.7 g guanidine carbonate is dissolved in 1400 g of water at 200 (saturated solution).

2302.8 g thioglycolic acid at 80% are poured slowly as a function the degassing of carbon dioxide (foam formation). The reaction is not exothermic.

The final limpid, colourless solution obtained is degassed under vacuum and concentrated to a final weight of 3135 g.

This solution crystallizes with caking (hygroscopic white crystals).

### EXAMPLE 8

### Preparation of Diguanidine Thioglycolate

2350 g of ice is added to a solution of 1801.7 g guanidine carbonate in 1400 g of water. This mixture is then added to the guanidine thioglycolate obtained in the previous example.

954.2 g of 97% calcium hydroxide at is added slowly under stirring to the previous mixture (exothermic reaction). The temperature is maintained below 20° C. during the reaction carried out under stirring for 4 hours. The pH is then 11.52 at 25° C.

The calcium carbonate formed is filtered through sintered glass under vacuum and then washed with 1000 g water.

The filtrate obtained, which is limpid and slightly pink (due to traces of iron), is concentrated under vacuum to a final weight of 5492 g.

The diguanidine thioglycolate crystallizes (white, slightly pink crystals). The colouration is eliminated by washing with 500 ml of iced water. White crystalline diguanidine thioglycolate is obtained

### EXAMPLE 9

### Preparation of Guanidine Gluconate

1000 g of 50% gluconic acid solution is placed in a 5-litre glass reactor under stirring.

229.6 g of anhydrous guanidine carbonate is added slowly to allow regular release of the carbon dioxide formed. The final weight at the end of the operation is 1171 g.

The solution obtained is then concentrated and degassed under vacuum to a final weight of 867 g.

The final composition is 57.7% gluconic acid and 13.3% guanidine base. The pH obtained is 5.376 at 25.9° C.

The guanidine gluconate crystallizes slowly from this very viscous solution (white crystals).

### EXAMPLE 10

### Preparation of Guanidine Salicylate

180.2 g of guanidine carbonate is dissolved in 695 g water and placed under stirring in a 5-litre glass reactor.

276.3 g salicylic acid is added slowly at a temperature of 40° C.

The solution is then degassed under vacuum, which causes sudden crystallisation of the guanidine salicylate.

The guanidine salicylate is then filtered through sintered glass and washed with 100 ml water (white crystals).

1. Rapid-action depilatory composition comprising
(1) at least one bisulphite and/or sulphite compound selected from the group consisting of:
(a) bisulphites or sulphites of potassium, calcium, lithium, barium or strontium;
(b) bisulphite or sulphite alkaline salts with a strong organic base, having a pK_{B} of the order of approximately 6 to 13.5; and
(c) bisulphitic aldehyde or ketone compounds, and
(2) an organic reducing agent,
in a cosmetically-acceptable medium.

2. Composition according to claim 1, characterized in that its acid components are in the form of a salt with a strong organic base.

3. Composition according to claim 2, characterized in that said strong organic base has a pK_{B} of the order of approximately 6 to 13.5.

4. Composition according to claim 2, characterized in that said strong organic base is guanidine, hydrazine or hydroxylamine.

5. Composition according to claim 1, characterized in that said bisulphite and/or sulphite compound is replaced by sulphur dioxide SO₂.

6. Composition according to claim 1, characterized in that it comprises from 0.1% to 25% of the bisulphite and/or sulphite compound, by weight of the total composition.

7. Composition according to claim 6, characterized in that it comprises 1% to 10% of bisulphite and/or sulphite compound, by weight of the total composition.

8. Composition according to claim 1, characterized in that the organic reducing agent is selected from the group consisting of dithiothreitol, thioglycerol, thioglycol, thiosalicylic acid and its salts, N-acetyl-L-cysteine, NaHSO₃, LiS, Na₂S, K₂S, MgS, CaS, SrS, BaS, (NH₄)₂S, sodium 6-8-dithiooctanoate dihydrolipoate, sodium 6-8-dithiooctanoate, hydrogen sulphide salts, mercaptocarboxylic acids of formula HS—(CH₂)ₙ—COOH in which n is an integer from 1 to 4 and their salts, thiomalic acid and its salts, thiomaleic acid and its salts, thiolactic acid and its salts, dihydrolipoic acid and its salts, homocysteine, cysteine, cysteamine, glutathione, thioglycolamide, 1-,3-dithiopropanol, keratinase, hydrazine, hydrazine sulphate, hydrazine disulphate, triisocyanate and guanidine.

9. Composition of claim 8, characterized in that the organic reducing agent is selected from the group consisting of hydrazine, hydroxylamine, thiourea, thioacetamides and mercaptocarboxylic acids of formula HS—(CH₂)ₙ—COOH in which n is an integer from 1 to 4 and their salts.

10. Composition according to claim 1, characterized in that the organic reducing agent is a mercaptocarboxylic acid of formula HS—(CH₂)ₙ—COOH in which n is an integer from 1 to 4 in the form of a salt with a strong organic base.

11. Composition according to claim 1, characterized in that the organic reducing agent is thioglycolic acid or a salt thereof.

12. Composition according to claim 11, characterized in that the organic reducing agent is guanidine thioglycolate.

13. Composition according to claim 1, characterized in that the organic reducing agent is present in an amount of 0.2% to 5% by weight of the total composition.

14. Composition according to claim 1, characterized in that it also comprises at least one compound selected chosen from the group consisting of
surface-active agents,
anti-irritant agents,
agents which accelerate the reduction reaction of keratin,
pH regulating agents, and
sequestering agents of monovalent or divalent cations.

15. Composition according to claim 14, characterized in that the penetration agent is sodium, potassium, calcium or strontium lauryl sulphate, or a lauryl sulphuric acid salt with a strong organic base.

16. Composition according to claim 15, characterized in that the sequestrating agent is sodium gluconate or a gluconic acid salt with a strong organic base.

17. Composition according to claim 10, characterized in that said strong organic base has a pK_{B} of the order of approximately 6 to 13.5.

18. Composition according to claim 17, characterized in that said strong organic base is guanidine, hydrazine or hydroxylamine.

19. Composition according to claim 1, characterized in that it also comprises at least one compound selected from the group consisting of preservatives, antioxidants and perfumes.

20. Composition according to claim 1, characterized in that it has a pH between 9 and 12.7, optionally of the order of 12.3.

21. Composition according to claim 1, characterized in that it is in the form of a gel, a foam, an emulsion or a cream.

22. Method for the preparation of a composition according to claim 1, characterized in that it comprises the steps of:
preparing a water-in-oil emulsion which does not contain the organic reducing agent and the sulphite or bisulphite compound,
preparing an aqueous suspension containing the organic reducing agent and the sulphite or bisulphite compound,
introducing said suspension into the oil-in-water emulsion, and
subjecting the mixture to shearing.

23. Method for removing superfluous hair, comprising the steps of:
applying a sufficient quantity of depilatory composition as described in claim 1 onto a skin surface comprising said hairs, then
removing said composition by wiping or rinsing, this action having the effect of simultaneously removing the hairs

24. Method according to claim 23, characterized in that the wiping or rinsing is carried out at the end of a period ranging from approximately 1 minute to 5 minutes after the application of the depilatory composition.

25. Method for the preparation of a strong organic base salt, characterized in that it comprises the steps of
reacting a strong acid with a strong organic base carbonate, and
degassing the carbon dioxide formed.

26. Method according to claim 25, characterized in that said strong acid has a pK_{A} less than 6.

27. Method according to claim 25, characterized in that the strong organic base used is guanidine, hydrazine or hydroxylamine.

28-29. (canceled)

30. A compound in the form of a salt of a strong organic base selected from the group consisting of salts of strong organic bases of mercaptocarboxylic acid, gluconic acid, salicylic acid, lauryl sulphuric acid, hydrochloric acid and formic acid.

31. The compound according to claim 30 which is selected from the group consisting of guanidine thioglycolate, diguanidine thioglycolate, guanidine gluconate, guanidine salicylate, guanidine lauryl sulphate, guanidine hydrochloride and guanidine formate.
